(19) United States
(12) Patent Application Publication
(10) Pub. No.: US 2025/0152318 A1
(43) Pub. Date: May 15, 2025

(54) SYSTEMS, METHODS, AND APPARATUS FOR LIGHT CURING WITH AN INTRAORAL SCANNER

(71) Applicant: ALIGN TECHNOLOGY, INC., San Jose, CA (US)

(72) Inventors: Igor MAKIEWSKY, Rama Gan (IL); Ofer SAPHIER, Rehovot (IL)

(73) Assignee: ALIGN TECHNOLOGY, INC., San Jose, CA (US)

(21) Appl. No.: 18/948,374

(22) Filed: Nov. 14, 2024

Related U.S. Application Data
(60) Provisional application No. 63/599,289, filed on Nov. 15, 2023.

(51) Int. Cl. A61C 13/15; A61B 1/00; A61B 1/24

(52) U.S. Cl. CPC A61C 19/004; A61B 1/00172; A61B 1/24

(57) ABSTRACT

A system for curing flowable dental material may include an intraoral scanner configured to capture 3D data, a dental-material-curing light source configured to emit curing light from the probe, a processor, and memory. The memory may include instructions that when executed by the processor cause the system to receive a 3D model of the dentition of the patient from a dental treatment plan, receive scan data of the dentition of the patient, and determine, while receiving the scan data, an area of illumination by the dental-material-curing light source with respect to a dentition of a patient, based on the received scan data.

### RELATED APPLICATIONS

This application claims the benefit under 35 U.S.C. §119(e) of U.S. Provisional Patent Application No. 63/599,289, filed Nov. 15, 2023, and titled “SYSTEMS, METHODS, AND APPARATUS FOR LIGHT CURING WITH AN INTRAORAL SCANNER,” which is incorporated, in its entirety, by this reference.

### BACKGROUND

The present disclosure is generally related to using and curing flowable dental materials, such as dental resins, with an intraoral scanner.

Dental treatments may involve the use of resins for filings, attaching restorative prosthetics to teeth, forming attachments, and other uses. Some treatments and uses may be less than ideal. Many resins used in dental applications are cured using light. The light may be of a wavelength that may cause harm to users. For example, overexposure to the light may cause damage to a dental professional's eyes. Inadequate curing may result in premature failure of dental prosthetics and orthodontic attachments. Light curing parameter, such as intensity and time, are a one-size-fits-all to limit exposure and achieve an appropriate cure. However, such parameters can cause inadequate curing and expose users to more potentially harmful light, may result in long cure times to ensure the resin is cured, uses a dedicated curing instrument.

In light of the above, improved devices and methods that overcome at least some of the above limitations of the prior devices and methods would be helpful.

### SUMMARY

Embodiments of the present disclosure provide improved dental resin curing using intraoral scanning systems and methods to provide more accurate dosing of curing light and limiting user exposure to the curing light.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIGS. 1A and 1B show an intraoral scanner with a dental material curing light source, in accordance with some embodiments;

FIG. 2 shows an intraoral scanning system, in accordance with some embodiments;

FIG. 3 shows a method of using an intraoral scanner with a dental material curing light source, in accordance with some embodiments;

FIGS. 4A, 4B, 4C, and 4D show aspects of calibration of the field of exposure of the dental material curing light source with the field of view of the intraoral scanner, in accordance with some embodiments;

FIG. 5 shows aspects of determining curing parameters of a dental material, in accordance with some embodiments;

FIG. 6 shows dental material curing feedback provided to a user, in accordance with some embodiments;

FIG. 7 shows aspects of evaluating the curing process, in accordance with some embodiments;

FIG. 8 shows a process of curing an attachment, in accordance with some embodiments;

FIG. 9 shows a process of curing and modifying a filling for occlusal contacts, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The methods, apparatus, and systems disclosed herein are well suited for combination with prior devices such as intraoral scanners, for example the iTero system commercially available from Align Technology, Inc.

The presently disclosed methods and systems are well suited for combination with prior approaches to scanning intraoral structures and curing dental material, such as generating three-dimensional models of a patient's dentition, forming fillings and attachments, and bonding prosthetics.

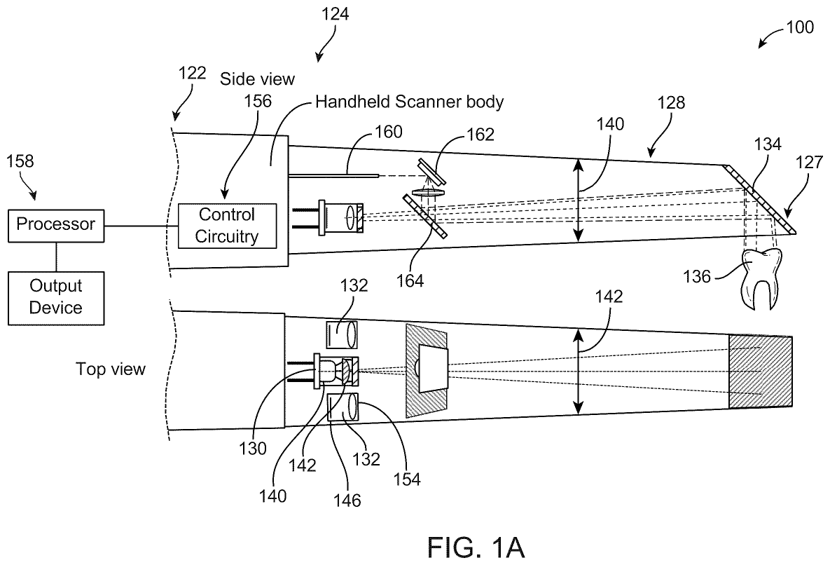

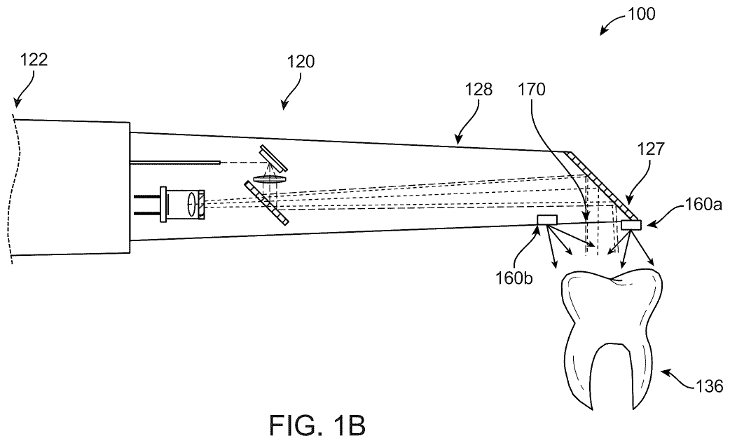

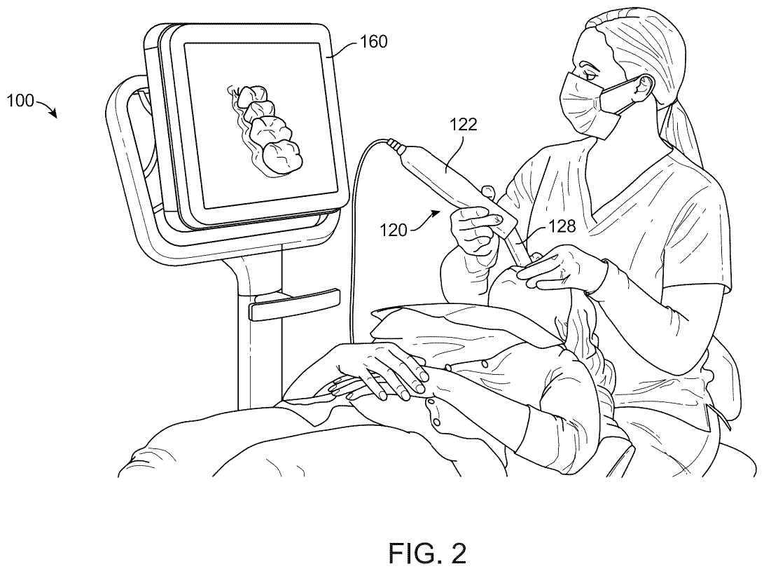

Reference is now made to FIGS. 1A, 1B, and 2, which are a schematic illustrations of an intraoral scanning system 100 with light curing, in accordance with some embodiments of the present invention. intraoral scanning system 100 comprises an elongate handheld wand 122 that has a probe 128 at distal end of the handheld wand 122. Probe 128 has a distal end 127 and a proximal end 124. As used herein, the proximal end of the handheld wand is the end of the handheld wand that is closest to a user's hand when the user is holding the handheld wand in a ready-for-use position and the distal end of the handheld wand is the end of the handheld wand that is farthest from the user's hand when the user is holding the handheld wand in a ready-for-use position.

In some embodiments, an intraoral scanner may include an intraoral imaging system including a structured light projector 130 disposed in proximal end 124 of probe 128 along with one or more light field cameras 132 also disposed in proximal end 124 of probe 128. A mirror 134 may be disposed in distal end 127 of probe 128. Structured light projector 130 and light field camera 132 are positioned to face mirror 134, and mirror 134 is positioned to reflect light from structured light projector 130 directly onto an object 136 being scanned and reflect light from object 136 being scanned into light field camera 132.

Although an embodiment of the intraoral 3D scanner with light curing is provided in FIG. 1A, light curing may be incorporated into a 3D scanning probe using many different types of 3D scanning hardware and software. For example, the 3D scanning system may be a confocal 3D scanning system, a photogrammetry scanner, or other 3D scanning system type.

Structured light projector 130 includes a light source 140. In some applications, structured light projector 130 focuses light from light source 140 at a projector focal plane that may be located external to the probe and at an object to be scanned 136. Structured light projector 130 may have a pattern generator 142 that is disposed in the optical path between light source 140 and the projector focal plane. Pattern generator 142 generates a structured light pattern at projector focal plane 138 when light source 140 is activated to transmit light through pattern generator 142.

Light field camera 132 has a light field camera sensor 146 that comprises an image sensor comprising an array of pixels, e.g., a CMOS image sensor, and an array of micro-lenses disposed in front of image sensor 148 such that each micro-lens is disposed over a sub-array of sensor pixels. Light field camera 132 additionally has an objective lens 154 disposed in front of light field camera sensor 148 that forms an image of object 136 being scanned onto light field camera sensor 146.

Intraoral scanner 100 may include control circuitry 156 that drives structured light projector 130 to project a structured light pattern onto object 136 outside handheld wand 122, and drives light field camera 132 to capture a light field that results from the structured light pattern reflecting off object 136. Using information from the captured light field, a computer processor 158 may reconstruct a three-dimensional image of the surface of object 136 and may output the image to an output device 160, e.g., a monitor. It is noted that computer processor 158 is shown herein, by way of illustration and not limitation, to be outside of handheld wand 122. In some embodiments, computer processor 158 may be disposed within handheld wand 122.

The structured light scanning system may generate point clouds representing the three-dimensional surface of the object 136 being scanned. The structured light service may generate up to 60 frames per second of point cloud data that may be used to generate a three-dimensional model of the surface of the object 136 being scanned. In some embodiments, the point cloud data may be used to determine the position and orientation of the scanning wand with respect to the intraoral structure of the object 136 being scanned.

In some embodiments, the structured light scanning system may also capture the color of the surfaces of the object 136. For example, in some embodiments the structured light source may be a white light source and the light field camera 132 may record the color of the surface of the object 136 based on the light reflected from the object.

Light-curing of dental resins involves using a special type of light to initiate and accelerate the polymerization of dental materials, such as flowable dental materials including resin-based dental materials, dental composite filling material, and other light-curable materials. Light-curing resins allow for controlled setting of the dental material. A dentist can shape the resin as desired while it remains in a malleable state and then quickly harden it using a curing light.

Dental resins may be made of monomers, such as monomers based on methacrylate or similar compounds, photoinitiators, and other additives. The photoinitiators are molecules that can absorb light and produce reactive species that can initiate the polymerization of the monomers. When the dental resin is exposed to a specific wavelength of light, for example blue light, with a wavelength around 400-500 nm, the photoinitiators in the resin absorb this light and become activated. Upon activation, the photoinitiators produce reactive species, such as free radicals. The free radicals then react with the monomers in the resin. This starts a reaction where monomers link together to form long polymer chains, converting the liquid or semi-solid monomer resin into a hardened polymer solid though a process called polymerization.

The light used to cure dental resins may be provided by a specialized dental curing light source. The light source may produce light at the wavelengths that can activate the photoinitiators in the dental resins. The light source may be Light Emitting Diode (LED). LED curing lights have increased efficiency and reduced heat production as compared to other light sources, such as halogen light sources. The light source may be capable of emitting light of various wavelengths or wavelength ranges. The wavelength or wavelength range of light emitted by the light source may be selected prior to or during the curing process, for example, based on the resin material to be cured by the curing light source.

The depth and shape of the resin applied to a tooth plays a role in the curing process. Less light may penetrate deeply into the resin than the amount of light that penetrates into the shallow layers or that falls on the surface of the light curable material. This depth of penetration and amount and rate of curing is influenced by the intensity and wavelength of the curing light, the composition and opacity of the dental resin, the angle of incidence of the light, and the duration of light exposure. In situations where a thick layer of material is to be cured, resin may be applied and cured in incremental layers to ensure complete polymerization. As discussed herein, systems and methods disclosed herein enable reliable curing or resin and light curable materials, including deep into a resin in thick light curable material applications.

The intraoral scanner probe 128 may include a resin curing light source 160. The light source may be a blue light LED. In some embodiments, the light source may emit light in a wavelength between 400 and 500 nm. The light from the light source 160 may be reflected off of one or more mirrors 162, 164 towards the mirror 134 at the distal end 127 of the probe 128. The light may exit the distal end of the probe and illuminate the dental object 136.

In some embodiments, optical elements other than mirrors or in addition to mirrors, such as optical fibers may be used to direct the light towards the dental object.

In some embodiments the light source may include a plurality of light sources. For example, the light source may be a plurality of LEDs or a two-dimensional array of light sources. In some embodiments, the light source may include a mask such as an LCD including a plurality of pixels arranged in a two-dimensional array. Activation and deactivation of the pixels in the array may control the amount and area of the light exiting the probe 128.

In some embodiments, curing light may be provided by the light sources used for 3D scanning. For example, light from the structured light projector 130 may be used for curing the resin. In some embodiments, light from light sources other than those used for 3D scanning may be used to cure the resin.

With reference to FIG. 1B, the probe 128 may include one or more light sources 160 that are located at the distal end of the probe. In some embodiments, a light source 160 may be located at an exterior of the probe 128.

In some embodiments, the light from the light sources may be emitted in proximal or distal directions. For example light source 160a emits light in a predominantly proximal direction while light from light source 160b emits light in a predominantly distal direction. Light source 160a may be located distal to the exit window 170 of the intraoral scanner. The exit window 170 is the window through which the intraoral scanner images the patient's teeth in order to generate a three-dimensional model of the patient's teeth. Light source 160b may be located proximal to the exit window 170 of the intraoral scanner.

Intraoral scanner 100 may include control circuitry 156 that drives the light sources 160 to project the light from the light source onto the dental object 136. Using calibration information from the alignment of the light source with intraoral scanning system, as discussed herein, and a computer processor 158 the amount of resin curing light received at each location on the surface of the dental object may be determined. The processor 158 may further use curing computations based on the properties of the dental object 136, such as the light absorption and scattering properties, the thickness, the geometry, and other properties, to determine the amount of curing that is occurring or has occurred for resin on the dental object 136.

In some embodiments, the intraoral scanner may scan the surface of the dentition while emitting light to cure the resin. Light from the curing process may be reflected into the 3D scanning system. This light may interfere with the capture of 3D scanning data. In some embodiments, a filter, such as a diachronic filter may be placed between the scanning imaging sensor and the object being scanned to filter out the wavelengths of light used for curing the resin. In some embodiments, scanning and curing may take place in an alternating or interleaved manner. For example, curing light may be turned on and emitted for a first period of time and then turned off. Then 3D scanning may take place during a second period of time. In some embodiments, the time for light curing may be between 3 times and 50 times as long as the time for 3D scanning, preferably between 5 times and 20 times. For example, a duty cycle of light curing may be between 50 and 99.5%, preferably between 80% and 95%. In some embodiments, such as when the 3D scanning imaging camera uses a global shutter, curing light may be emitted when the shutter is off, for example during the read phase of the imaging device operation or not during the exposure phase of the imaging device operation. In some embodiments, the time for light curing may at least 3 to 50 times as long as the time for 3D scanning, preferably at least 5 times to 20 times.

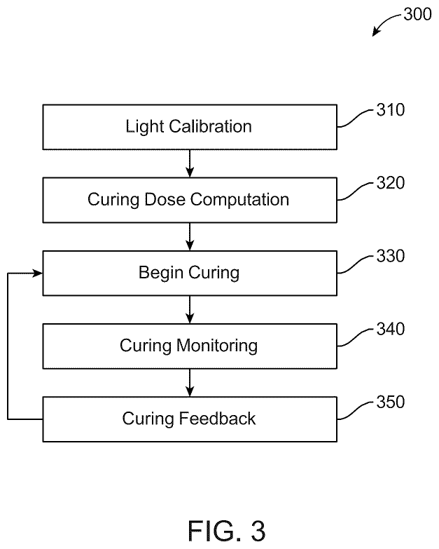

FIG. 3 depicts a method 300 of using a light-curing and intraoral scanning system 100 to monitor and provide feedback with respect to the curing of dental resins and other curable dental materials. The method 300 may include calibrating the curing light emitted from the light source with respect to the field of view of the intraoral scanner at block 310, computing the curing dose for curing the resin at block 320, beginning the curing process at block 330, monitoring the curing process at block 340, and providing curing feedback at block 350.

At block 310 the field of view of the curing light emitted from the light source is calibrated with respect to the field of view of the intraoral scanner. The light source may not be emitted uniformity. In some embodiments, the light may suffer from intensity fall off at the edges or other optical effects that may cause the intensity of the light to vary over the area of illumination of the light. In some embodiments, the light emitted from the light source may vary due to the use of multiple light sources such as multiple LEDs with overlapping areas of light projection. In some embodiments, the area over which the curing light is projected may not be coincident with the area of the field of view of the intraoral scanning system. Calibration of the light source and the field of view of the intraoral scanner allows the system to determine the location and corresponding intensity of the curing light on the dental object.

In some embodiments, the calibration of the light intensity may be performed using an external light sensor, such as a camera, that takes images of the light on a surface and may calibrate the light source based on the recorded intensity of the light on the surface. In some embodiments, the calibration may be performed with the camera or image sensor of the intraoral scanner.

When the field of view of the intraoral scanner and the area of light projection are calibrated to each other, the system may use the intraoral scanner to determine the position and orientation of the probe and then, based on the position and orientation of the probe, may determine the illumination location and intensity provided by the curing light source.

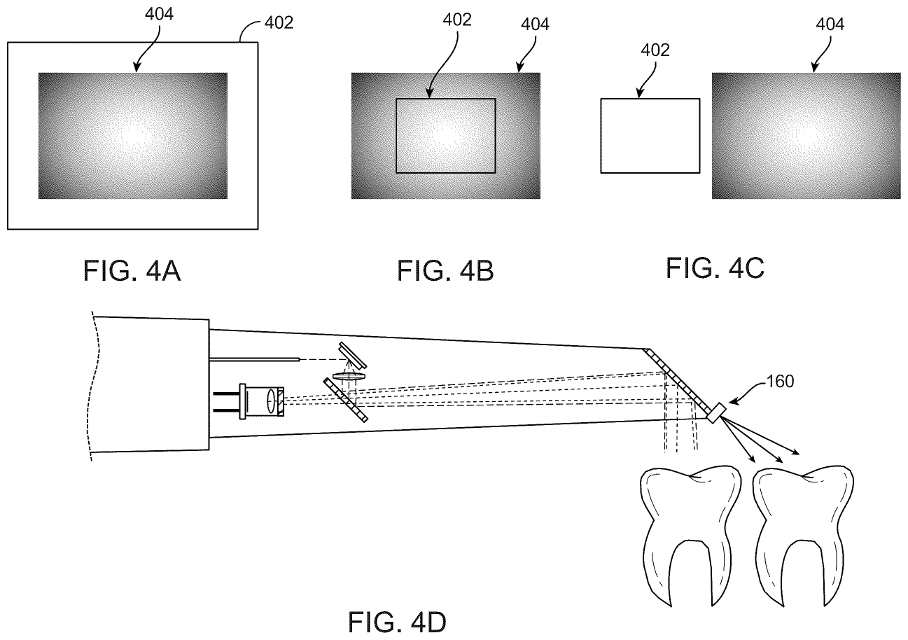

FIGS. 4A, 4B, 4C, and 4D depict various arrangements of the 3D scanning field of view and a light projection area that a calibration, such as the calibration described in block 310, may calibrate for. FIG. 4A depicts an embodiment wherein the field of view 402 is greater than the light projection area 404. As shown in FIG. 4A, the light projected by the light source results in uneven illumination over the light projection area 404. The light illumination is most intense in the center of the light projection area 404 and falls off towards the perimeter and corners of the light projection area 404 before falling off to little or nothing before reaching the edge of the field of view of the intraoral scanner 402. After calibration, the system will be able to determine the amount of light illuminating the particular area of the dental object based on the 3D structure scanned by the 3D scanner and will know that the field of view of the 3D scanner exceeds the area of illumination 404 of the light source.

FIG. 4B depicts an embodiment wherein the field of view 402 is less than the light projection area 404. As shown in FIG. 4B, the light projected by the light source results in uneven illumination over the light projection area 404. The light illumination is most intense in the center of the light projection area 404 and falls off towards the perimeter and corners of the light projection area 404 before falling off to little or nothing beyond the edge of the field of view of the intraoral scanner 402. After calibration, the system may determine the amount of light illuminating the particular area of the dental object based on the 3D structure scanned by the 3D scanner and determine that the field of view of the 3D scanner is less than the area of illumination 404 of the light source.

FIG. 4C depicts an embodiment wherein the field of view 402 does not overlap with the light projection area 404. As shown in FIG. 4C, the light projected by the light source results in uneven illumination over the light projection area 404. The light illumination is most intense in the center of the light projection area 404 and falls off towards the perimeter and corners of the light projection area 404 before falling off to little or nothing. After calibration, the system may determine the amount of light illuminating the particular area of the dental object based on the 3D structure scanned by the 3D scanner and will know that the field of view of the 3D scanner does not overlap with the area of illumination 404 of the light source.

FIG. 4D depicts an example wherein the field of view 402 of the intraoral scanner does not overlap with the area of illumination 404 of the light sources. As shown, the light source 160 projects light on a distal dental object while the intraoral scanner scans a more proximal dental object.

The calibration may also calibrate based on the distance between the probe and the dental object, the angle of the probe with respect to the surface of the dental object, and other factors. In some embodiments, the calibration may be based on scanning and illuminating objects or surfaces of known sizes and shapes from known distances and angles.

With reference back to FIG. 3, at block 320 the curing dose for curing the resin is determined. The method may use the 3D geometry of the patient's detention, such as the size and shape of the dental prosthetic, the size and shape of the attachment, the geometry of the filling, etc., the type of resin, the thickness of resin, and other factors, to determine how much photonic energy should be given to each location to cure the resin. In some embodiments, the method may also consider tolerances for curing the resin. For example, the method may determine an amount of photonic energy to cure the resin and add an additional amount of photonic energy as a tolerance or safety factor such that the light curable material is more likely to be cured with the cure dose. In some embodiments, the tolerance may be based on the 3D geometry of the patient's detention, such as the size and shape of the dental prosthetic, the size and shape of the attachment, the geometry of the filling, etc., the type of resin, the thickness of resin, and other factors. In some embodiments, the photonic energy may be determined based on the location of a volume of resin. For example, the photonic dose may be determined for each of a plurality of volumes of resin that makes up the total volume of resin used. For example, a model of the volume of resin may be divided into a plurality of voxels, each voxel may be assigned a cure dose. In some embodiments, the photonic energy reaching each voxel or portion of the volume of the resin may be determined based on an amount of photonic energy that illuminates the surface of the resin or dental object that would reach and cure the whole volume of resin. In some embodiments, the intensity may be based on light illuminating from the surface normal of the resin and/or dental object.

In some embodiments, the determination may be based on ray tracing simulations of the light source illuminating the resin and dental objects.

In some embodiments, the determining the curing dose may include taking into account the absorption and scattering of the curing light as it passes through the resin and other dental structures, such as a dental prosthetic, an attachment tray, or other object.

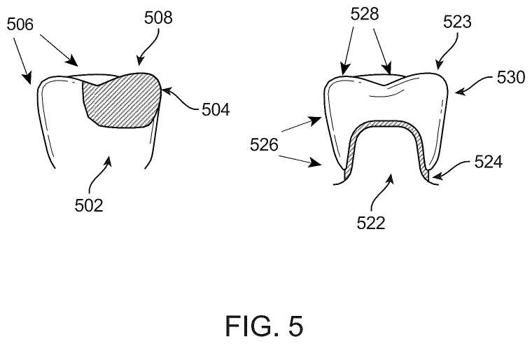

FIG. 5 depicts two examples of resin and dental structures for computing the curing dose. Tooth 502 may have had a cavity that was removed and the dentist elected to fill the cavity with a filling. The geometry of the tooth 502 may be determined based on a 3D scan of the tooth, such as by using the intraoral scanner. The resin 504 that makes of the filling may be modeled on the model of the tooth. Based on the combination of the tooth model 502 and the filling model 504, at block 320, the curing dose, including the amount of photonic energy for curing the resin 504 may be determined.

The dose may be determined based on one or more positions and orientations of a light source. For example, the dose may be determined based on a position and orientation 506. From this position and orientation the light from the light source may pass through a portion of the tooth before illuminating the resin. The determination may account for the light absorption and scattering as the light passes through the tooth in determining the rate at which photonic energy cures the resin from the position and orientation. In addition, some light may directly illuminate the surface of the resin, but does so at a large angle with respect to the normal angle of the surface of the resin. The determination may account for this angle in determining the rate at which photonic energy cures the resin.

The dose may be determined based on a position and orientation 508. From this position and orientation the light from the light source may directly illuminate the surface of the resin and pass through a thickness of the resin to reach the bottom of the resin making up the filling. The determination may account for the light absorption and scattering as the light passes through the resin in determining the rate at which photonic energy cures the resin from this position and orientation. Some light may illumination the surface of the resin from an angle normal to the surface while other locations on the surface may be illuminated at an angle with respect to the surface normal. The determination may account for the angle with respect to the surface normal in determining the rate at which photonic energy cures the resin.

Tooth 522 may be a tooth that was prepared to receive a prosthetic, such as a crown 523. The geometry of the tooth 522 may be determined based on a 3D scan of the tooth, such as by using the intraoral scanner. The geometry of the crown may be based on a 3D scan of the crown or a 3D model of the crown. Resin 524 may be used to couple the crown 523 to the prepared tooth 522. Based on the combination of the tooth model 522, the crown model 523 and a model of the resin 524, which may be based on the shape of the tooth model 522 and the crown model 523, at block 320, the curing dose, including the amount of photonic energy for curing the resin 524 may be determined.

The dose may be determined based on one or more positions and orientations of a light source. For example, the dose may be determined based on a position and orientation 526. From this position and orientation the light from the light source passes through a portion of the crown before illuminating the resin. The determination may account for the light absorption and scattering as the light passes through the crown in determining the rate at which photonic energy cures the resin from the position and orientation. Resin on the left side of the tooth 522 may receive the curing light though a relatively thin portion of the crown and at a position and angle approximately aligned with the surface normal of the crown at that location. The resin on the left side of the tooth 522 may receive a relatively high percentage of the photonic energy illuminating the crown from this position and orientation while resin on the top and right sides may receive a relatively low percentage of the photonic energy illuminating the crown from this position and orientation.

The dose may also be determined based on a position and orientation 528. From this position and orientation the light from the light source passes through a portion of the crown before illuminating the resin. The determination may account for the light absorption and scattering as the light passes through the crown in determining the rate at which photonic energy cures the resin from the position and orientation. Resin on the top of the tooth 522 may receive the curing light though a relatively thick portion of the crown and at a position and angle approximately aligned with the surface normal of the crown at that location. The resin on top of the tooth 522 may receive a relatively high percentage of the photonic energy illuminating the crown from this position and orientation while resin on the left and right sides may receive a relatively low percentage of the photonic energy illuminating the crown from this position and orientation.

The dose may also be determined based on a position and orientation 530. From this position and orientation the light from the light source passes through a portion of the crown before illuminating the resin. The determination may account for the light absorption and scattering as the light passes through the crown in determining the rate at which photonic energy cures the resin from the position and orientation. Resin on the top and upper right side of the tooth 522 may receive the curing light though a relatively thin portion of the crown and at a position and angle approximately that is not aligned with the surface normal of the crown at that location. The resin on top and upper right side of the tooth 522 may receive a relatively high percentage of the photonic energy illuminating the crown from this position and orientation, however this amount may be less than what the left side receives from position 526 and what the top receives from position 528. Resin on the left and lower right sides may receive a relatively low percentage of the photonic energy illuminating the crown from this position and orientation. The lower right side may receive less photonic energy due the angle of the light illumination with respect to the surface normal and the increased thickness of the crown that the light passes through at this angle before reaching the resin 524.

The computed does may be a minimum expected dose. For example, the measurements of probe position and orientation, amount of curing illumination or light energy, the material properties of the resin, and the other factors discussed herein may not be perfectly precise. In other words, there may be an error or uncertainty associated with the factors. When computing the dose received by the resin and/or the state of curing of the resin, the lower bounds of errors or uncertainty of the factors with respect to curing does may be used in the dose calculation.

Returning at FIG. 3, at block 330 the curing process begins. During the curing process, light from the curing light source illuminates the dental structure while the scanner scans the surface of the dental structure.

At block 340, the curing process is monitored. Monitoring the curing process may include monitoring the light output by the curing light source, such as by imaging and mapping the light output on the dental structures. The actual light output by the curing light source may be compared to calibration data and if it varies from the expect light output, based on the calibration data, the photonic energy calculation and the curing state may be updated based to account for changes between the calibrated light output and the actual light output. If the light output varies beyond a threshold of intensity variance between the actual measured light output and the expected light output based on the calibration data, the system may provide feedback to the user, such as feedback to recalibrate the probe.

In some embodiments, monitoring the curing process may include monitoring for changes in the 3D structure of the patient's dentition. For example, in some embodiments, resin may change volume as it cures. Some resins may shrink while other resins may expand. During the curing process, the scanner may repeatedly scan the surface of the patient's dentition to determine the surface shape of the dentition. The system may compare the shape of the 3D surface, such as the shape and position of a crown as the resin cures. A change in the position of the crown during the curing process may indicate that the resin curing process is complete. If, based on the change of shape or position of the resin or crown, the curing process completes after an unexpected amount of photonic energy is used to cure the resin, the amount of photonic energy used to cure the resin may be used to update the calibration and computational models. For example, the parameters of the model, such as absorption, scattering, etc., may be altered to adjust for the actual amount of photonic energy used.

In some embodiments, the shape or position of the resin or a crown may change shape or position asymmetrically or in an unexpected way, such as in an unexpected shape or position that is different than a planned or expected shape or position. Such a change in shape or position may indicate that the resin has not cured as expected and, for example, a crown or other prosthetic may not be properly or completely bonded to the patient's anatomy or that internal stresses may exist in the crown or prosthetic which may lead to premature separation of the crown or prosthetic from the patient's anatomy. Feedback may be provided to the user regarding the unexpected change in shape or position, such as visual feedback or audible feedback.

Monitoring the curing process may include determining the location and intensity of the illumination provided by the curing light source adding or integrating the amount of photonic energy provided by the curing light source to the resin. The method may include receiving a three-dimensional model of the patient's dentition such as from a previously scanned model of the patient's teeth. In some embodiments during the monitoring, the intraoral scanner scans the patient's dentition or other intraoral structure and uses the three-dimensional data from the scan to determine the position and orientation of the probe 120. For example, the method may include aligning the three-dimensional data from the monitoring scan with a location and orientation on the previously scanned three-dimensional model. From the position and orientation of the probe 120, the position and orientation of the illumination of the light source may be determined based on the calibration discussed in block 310.

In some embodiments, the system may use the curing dose computations determined at block 320 along with the position and orientation of the probe determined in block 340 along with a calculated or known intensity provided by the curing light source to determine how much photonic energy is reaching the resin, such as each volume or voxel of resin or other dental material and to determine an amount of curing and the location of curing of the resin or other dental material.

The process may determine, during the curing process, the location and amount of curing. In some embodiments, the position, location, and intensity of the light source along with the 3D geometry of the patient's detention, such as the size and shape of the dental prosthetic, the size and shape of the attachment, the geometry of the filling, etc., the type of resin, the thickness of resin, and other factors, to determine the how much photonic energy is reaching each volume or voxel of the resin and the curing status of the resin. In some embodiments, the photonic energy may be determined based on the location of a volume of resin. For example, the photonic energy reaching the resin may be determined for each of a plurality of volumes of resin that makes up the total volume of resin used. In some embodiments, the photonic energy may be determined based on an amount of photonic energy that illuminates the surface of the resin or dental object that reaches and cures the whole volume of resin. In some embodiments, the intensity may be based on light illuminating from the surface normal of the resin and/or dental object.

In some embodiments, the determination may be based on ray tracing simulations of the light source illuminating the resin and dental objects during the curing process. Ray tracing may include light simulations of the external ray tracing, such as the tracing of light ray reflections off of the surface of objects and may also include internal ray tracing, such as tracing the light within a volume as it is absorbed, internally scattered, and emitted from an object.

In some embodiments, the determining the amount of photonic energy reaching and curing the resin during the curing process may include accounting for the absorption and scattering of the curing light as it passes through the resin and other dental structures, such as a dental prosthetic, an attachment tray, or other object.

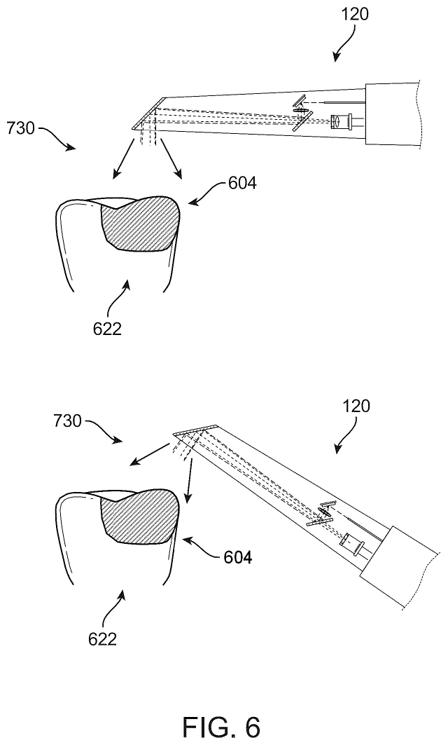

FIG. 6 shows two embodiments of the probe 120 emitting light 730 to cure resin 604 to fabricate a filling on a patient's tooth 622. At the left of FIG. 6 the probe 120 is located in the occlusal position and orientation with the light 730 emitting in a general direction towards the occlusal surface of the tooth 622. In order to estimate or otherwise determine how the resin 604 may be curing in response to the light, the intraoral scanner may first scan a portion of the patient's dentition such as a portion of the tooth 622 or dental structures near or adjacent to the tooth 622 in order to determine the position and orientation of the probe and the light source with respect to the resin 604. The position, location, and intensity of the light source along with the 3D geometry of the patient's detention, such as the size and shape of the dental prosthetic, the size and shape of the attachment, the geometry of the filling, etc., the type of resin, the thickness of resin, the wavelength of the curing light, the properties of the resin, including as the resin cures, including the light scattering, absorption, and resin response to each wavelength, and other factors, may be used to determine the how much photonic energy is reaching the resin and the curing status of the resin.

Determining how much photonic energy is reaching or has reached each portion or volume of the resin 604 may be based on how much light is illuminating the surface of the resin or other dental structure and at what angles of incidence. In some embodiments, determining how much photonic energy is reaching each portion or volume of the resin 604 may also be based on how much light is reflecting from the surface of the resin or other dental structure.

In some embodiments, determining how much photonic energy is reaching each portion or volume of the resin 604 may be based on a volumetric model of the patient's dentition and/or the resin. A volumetric model may take into account the light scattering and absorption properties of the material through the volume of material, such as the patient's dental structures and the resin. In some embodiments, the determination may be based on ray tracing simulations of the light source illuminating the volume of the resin and dental objects during the curing process.

In the embodiment shown on the upper portion of FIG. 6 the system may determine that the light is falling on the occlusal surface of the resin 604 and may calculate how much photonic energy is reaching each portion of the resin 604 from this position and orientation of the probe.

The lower portion of FIG. 6 depicts the probe 120 in a different position. This may be at a time after the curing shown on the upper portion, such as when the dentist moves the probe 120 to a more lingual side of the tooth 622 in order to provide more curing light and photonic energy to the lingual portions of the resin 604 that makes up with filling. In order to determine the amount of photonic energy reaching the resin and the amount of curing taking place, the intraoral scanner may first scan a portion of the patient's dentition such as a lingual portion of the tooth 622 or dental structures near or adjacent to the tooth 622 in order to determine the position and orientation of the probe and the light source with respect to the resin 604. The position, location, and intensity of the light source along with the 3D geometry of the patient's detention, such as the size and shape of the dental prosthetic, the size and shape of the attachment, the geometry of the filling, etc., the type of resin, the thickness of resin, and other factors, may be used to determine the how much photonic energy is reaching the resin and the curing status of the resin.

Determining how much photonic energy is reaching each portion or volume of the resin 604 may be based on how much light is illuminating the surface of the resin or other dental structure and at what angles of incidence. In some embodiments, determining how much photonic energy is reaching each portion or volume of the resin 604 may also be based on how much light is reflecting from the surface of the resin or other dental structure.

In some embodiments, determining how much photonic energy is reaching each portion or volume of the resin 604 may be based on a volumetric model of the patient's dentition and or the resin. A volumetric model may take into account the light scattering and absorption properties of the material through the volume of material, such as the patient's dental structures and the resin. In some embodiments, the determination may be based on ray tracing simulations of the light source illuminating the volume of the resin and dental objects during the curing process.

In the embodiment shown on the right side of FIG. 6 the system may determine that the light is falling on the lingual surface of the resin and the lingual portion of the occlusal surface of the resin 604 and may calculate how much photonic energy is reaching each portion of the resin 604 from this position and orientation of the probe.

The process may then add up the photonic energy received by each volume of resin for each of the time periods the wand was in the positions shown on each of the upper and power portions of the FIG. 6 in order to determine the total amount of photonic energy received by each volume of resin. In some embodiments, the photonic energy received by each volume of resin may be determined for a plurality of time intervals. For each time interval, the position of the wand and the light source is determined and then the amount of photonic energy received by the resin is determined. The amount of energy may be integrated over time to determine the total amount of energy received by each volume of resin.

The amount of energy used to cure the resin may be used to provide feedback to the user as to the estimated status of the curing operation.

In some embodiments, at block 350 feedback is provided to the user regarding the status of the curing process. The feedback may include visual, audio, or haptic feedback. Feedback may include feedback regarding the estimated curing status of the resin, the completion of the curing process, that the curing light is not illuminating the resin or that the curing light may be moved to provide increased illumination to the resin. In some embodiments, the curing at block 330, the monitoring at block 340, and the feedback at block 350 may repeat until the curing process is completed. For example, after providing feedback at block 350, the process may continue at block 330 again with additional curing, then to block 340 for an updated estimation of the cure of the resin, and then back to block 350 to display the updated feedback.

In some embodiments, the system may monitor the position and orientation of the probe and the illumination of the curing light during curing to provide additional curing and safety features. For example, in some embodiments, the system may monitor the position of the probe 120 by repeatedly using the scanner to scan portions of the patient's dentition such as a portion of the tooth 622 or intraoral dental structure in order to determine the position and orientation of the probe and the light 730 emitted by the light source with respect to the resin 604 as the probe moves over time. As discussed herein, the system may attempt to align the scanned 3D features with 3D features of an existing 3D model of the patient's dentition to determine the position and orientation of the probe and the light source.

In some embodiments, the system may being a curing process with the curing light source off while repeatedly scanning the intraoral structure until the system determines that the light source is in a position and orientation such that, when the curing light source is turned on, the curing light may illuminate the uncured resin. The system may then activate the curing light source.

In some embodiments, during curing, while the curing light source is active, the system may continue to monitor the position and orientation of the probe and the illumination of the curing light source by repeatedly scanning the intraoral structure until the system determines that the light source is in a position and orientation such that, the curing light source is not illuminating the resin or pointed in a direction outside the intraoral cavity or pointed in an unknown direction. When in such a situation, the curing light may be turned off and feedback, such as a visible (including light), audible, or haptic feedback may be provided to the dentist or operator.

The system may then continue to repeatedly scan the intraoral structure until the system determines that the light source is in a position and orientation such that, when the curing light source is turned on, the curing light may illuminate the uncured resin. The system may then activate the curing light source.

In some embodiments, the system may alter the illumination of the curing light source based on the monitored position and orientation of the probe 120 and the curing light source. For example, when the resin that is being cured is smaller than the illumination pattern or area of the curing light source, portions of the curing light source may be turned off or mased off.

In some embodiments, the curing light source may include a plurality of controllable light sources, the controllable light sources may be individually controllable or controllable in subgroups of lights, each subgroup including at least two light sources. The illumination pattern of each of the plurality of controllable light sources may be calibrated, as discussed above with respect to block 310. During curing, the system may repeatedly monitor the position of the probe 120 by repeatedly using the scanner to scan and image portions of the patient's dentition such as a portion of the tooth 622 or intraoral dental in order to determine the position and orientation of the probe and the light 730 emitted by the light source with respect to the resin 604 as the probe moves over time. As discussed herein, the system may attempt to align the scanned 3D features with 3D features of an existing 3D model of the patient's dentition to determine the position and orientation of the probe and the light source. The system may determine, based on the position and location of the probe and curing light source, along with a known position and shape of the resin, a portion of the illumination area provided by the curing light source that would illuminate the resin is located and portion of the illumination area provided by the light source that would not illuminate the resin. The system may then turn off light sources whose light does not illuminate the resin.

In some embodiments, the curing light source may include a controllable mask, such as a LCD screen or a digital micromirror device between the light source and the resin, the controllable mask may be controlled to block or mask off light that may not illuminate the resin or to permit light to pass through that would illuminate the resin. The illumination pattern of the mask may be calibrated during the calibration process, as discussed above with respect to block 310, such as by turning on and off pixels of the LCD screen and calibrating the illumination of the light source to account for the effect of each pixel or group of pixels. During curing, the system may repeatedly monitor the position of the probe 120 by repeatedly using the scanner to scan portions of the patient's dentition such as a portion of the tooth 622 or intraoral dental in order to determine the position and orientation of the probe and the light 730 emitted by the light source with respect to the resin 604 as the probe moves over time. As discussed herein, the system may attempt to align the scanned 3D features with 3D features of an existing 3D model of the patient's dentition to determine the position and orientation of the probe and the light source. The system may determine, based on the position and location of the probe and curing light source, along with a known position and shape of the resin, a portion of the illumination area provided by the curing light source that would illuminate the resin is located and portion of the illumination area provided by the light source that would not illuminate the resin. The system may then mask off a portion of the light source or light sources whose light does not illuminate the resin using the mask, such as by activating pixels of the LCD screen.

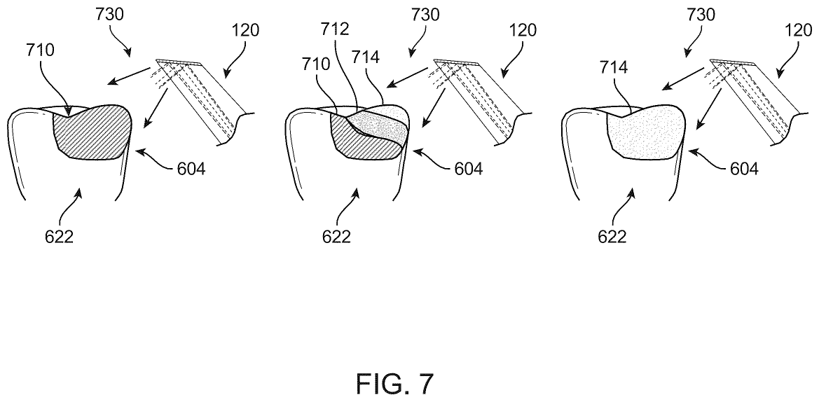

FIG. 7 depicts three stages of the curing process and the feedback provided to the user during the process. At the left side of FIG. 7, a first stage of the curing process is depicted. At this stage the curing process has just started. The feedback may use the information determined at block 340, such as the amount of photonic energy received at each volume of the resin and/or the estimated amount of resin curing for each volume. For example, the intraoral scanner 120 may first scan a portion of the patient's dentition such as a portion of the tooth 622 or dental structures near or adjacent to the tooth 622 in order to determine the position and orientation of the probe and the light 730 emitted by the light source with respect to the resin 604. The position, location, and intensity of the light 730 along with the 3D geometry of the patient's detention, such as the size and shape of the dental prosthetic, the size and shape of the attachment, the geometry of the filling, etc., the type of resin, the thickness of resin, and other factors, may be used to determine the how much photonic energy is reaching the resin and the curing status of the resin.

In some embodiments, determining how much photonic energy is reaching each portion or volume of the resin 604 may be based on a volumetric model of the patient's dentition and or the resin. A volumetric model may take into account the light scattering and absorption properties of the material through the volume of material, such as the patient's dental structures and the resin. In some embodiments, the determination may be based on ray tracing simulations of the light source illuminating the volume of the resin and dental objects during the curing process.

In the embodiment shown on the left side of FIG. 7 the system may determine that little to no curing of the resin 604 has taken place at this point in time and feedback 710, which may include a coloring the portion of the resin 604 with a first color, may be provided. In some embodiments, the feedback may be provided on a surface model of the resin. For example, the feedback may color the surface of the model based on the amount of curing throughout the volume of the resin. For example, the color of the surface of the resin may change based on the estimated cure state of the volume of resin, changing from red to yellow to green as curing progresses.

In some embodiments, the feedback may be provided in the form of one or more cross section of the volume of the resin. The cross section may be user selected, for example, an input may be received indicating the plane with which to make the cross section. In some embodiments, the resin may be sliced or cut into multiple cross sections, which may provide an exploded view of the volume of the resin and the feedback may be provided on each of the slices or cross sections. In some examples, the input may include one or more angles for the plane or cross section. In some embodiments, the feedback may be provided on a combination of an external surface and on a surface of a cross section through the internal volume of the resin. In some embodiments, the feedback may be made on a translucent volumetric model of the resin that includes colors or shading throughout the volume of the resin. In some embodiments, the feedback may include the resin with the least estimated amount of cured resin or the resin that has received the lowest dose of curing energy.

The curing process may continue and updated feedback may be provided as the curing process processes. For example, in the center of FIG. 7, the curing process has advanced. In the time between the state shown on the left side and the state in the center, the system has continued to monitor the position of the probe 120 by repeatedly using the scanner to scan portions of the patient's dentition such as a portion of the tooth 622 or dental structures near or adjacent to the tooth 622 in order to determine the position and orientation of the probe and the light 730 emitted by the light source with respect to the resin 604 as the probe moves over time. The position, location, and intensity of the light 730 for each time interval along with the 3D geometry of the patient's detention, such as the size and shape of the dental prosthetic, the size and shape of the attachment, the geometry of the filling, etc., the type of resin, the thickness of resin, and other factors, may be used to determine the how much photonic energy is reaching the resin for each time interval between the time depicted in left side of FIG. 7 and the center of FIG. 7. Based on the sum of the photonic energy received by each volume of resin over time, which may be determined by integrating the photonic energy received by each volume over time, the estimated amount of curing may be updated and feedback provided.

For example as depicted in the center of FIG. 7, the resin has been updated to show three different types of feedback. The substantially uncured portion of the resin 604 is highlighted in a first color, such as red to indicate that it remains substantially uncured. A portion of the resin 604 has begun curing or is an intermediate stage of curing and may be depicted with a second type of feedback 712, such as a second color, such as yellow. A portion of the resin 604 is also completed or substantially completed curing and may be depicted with a third type of feedback 714, such as a third color, such as green.

In some emblements, a threshold amount of estimated curing may be used to determine the feedback type. For example, when the estimated curing exceeds a first threshold, the feedback may change from the first feedback type 710 to the second feedback type 712. In some embodiments, when the estimated curing exceeds a second threshold, the feedback may change from the first feedback type 712 to the second feedback type 714. In some embodiments, additional thresholds and feedback types may be used. Although depicted as three different colors with distinct boundaries, in some embodiments, the colors may change gradually from one to another, such as in a gradient.

The curing process may continue and updated feedback may be provided as the curing process processes. For example, in the right of FIG. 7, the curing process has advanced to completion. In the time between the state shown in the center side and the state on the right side, the system has continued to monitor the position of the probe 120 by repeatedly using the scanner to scan portions of the patient's dentition such as a portion of the tooth 622 or dental structures near or adjacent to the tooth 622 in order to determine the position and orientation of the probe and the light 730 emitted by the light source with respect to the resin 604 as the probe moves over time. The position, location, and intensity of the light 730 for each time interval along with the 3D geometry of the patient's detention, such as the size and shape of the dental prosthetic, the size and shape of the attachment, the geometry of the filling, etc., the type of resin, the thickness of resin, and other factors, may be used to determine the how much photonic energy is reaching the resin for each time interval between the time depicted in center of FIG. 7 and the right of FIG. 7. Based on the sum of the photonic energy received by each volume of resin over time, which may be determined by integrating the photonic energy received by each volume over time, an estimated amount of curing may be updated and feedback provided.

For example, as depicted in on the right of FIG. 7, the resin has been updated to show feedback. The substantially cured portion of the resin 604 is highlighted in a color, such as green to indicate that the resin is substantially uncured.

In some emblements, a threshold amount of estimated curing may be used to determine the feedback type. For example, when the estimated curing exceeds a threshold, the feedback may change from the first feedback type to a final curing feedback type 714.

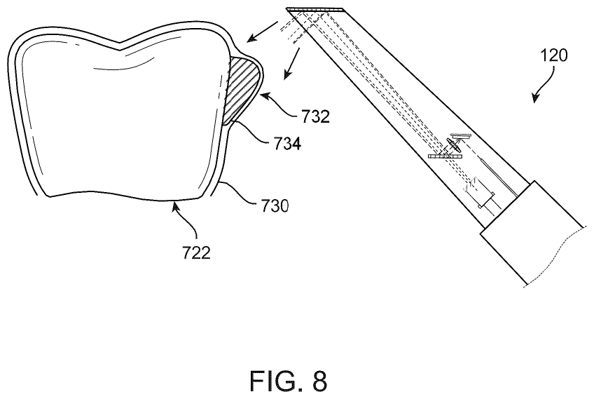

With reference to FIG. 8, the system 100 may be used to cure and bond attachments 734 to a patient's tooth using an attachment template 730. An attachment template 730 may be a sheet of polymer material formed into a shell having tooth receiving cavities shaped to receive the teeth of the patient. The attachment template 730 may include attachment wells 732. The attachment wells may be shaped according to the shape of a respective attachment, based on an orthodontic treatment plan. The attachment wells may be located on tooth receiving cavities according to the position of attachments specified by the treatment plan. In some embodiments, the attachment well may include uncured or only partially cured resin. During use, the attachment template 730 is placed over the patient's tooth 722 to place the attachment well and the resin within the well in the correct location as specified by the treatment plan.

The dentist may then use the probe with the scanner and curing light source, as described herein, to cure the resin in the attachment well. The system may monitor the curing process activating the curing light source or portions thereof, as discussed herein, such as described with respect to method 300. The system may also provide feedback on the curing process, as discussed herein, such as described with respect to method 300.

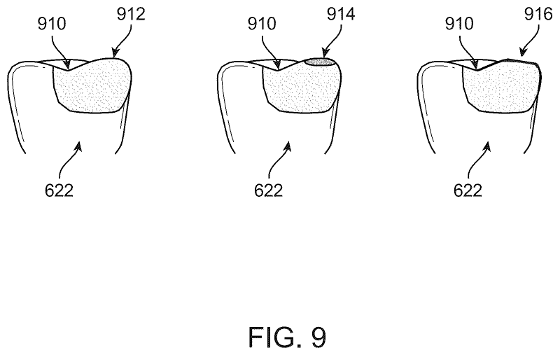

With reference to FIG. 9, a process for modifying a cured resin or light cured dental material is depicted. The tooth 922 may have had a cavity or other defect. A filling 910 may have been formed on the tooth 922 to rebuild the occlusal surface of the tooth 922. The filling may alter the occlusal contacts between the tooth 922 on a first jaw, such as a lower or upper jaw, and tooth or teeth that come into occlusion with tooth 922 of an opposing jaw, such as the other of the upper and lower jaw. The system may use an existing 3D model of the patient's dentition as a basis for determining whether the filling and the new occlusal surface formed by the filling improperly occludes with the opposing jaw forming a malocclusion. After forming the filling, the system may scan the filling and modify an existing 3D model of the patient's dentition with updated surface data that includes the new surface formed by the filling.

The system may then determine the extent of the occlusal contacts, such as by generating an occlusalgram that maps the location and magnitude of the occlusal contacts. They system may determine that a portion 912 of the resin extends beyond a desired height and may cause improper occlusal contacts between the lower and upper arch. Feedback as to the location of the portion 912 may be provided to the dentist, such as by highlighting the portion 912 or coloring the portion 912.

The dentist may then use a tool, such as a burr tool to remove material from the filling or resin volume 910. After removing the material, the dentist may rescan the location. The system may receive scan data from the scanned filling and modify an existing 3D model of the patient's dentition with updated surface scan data that includes the new surface formed by the material removal operation. The system may then determine the extent of the occlusal contacts, such as by generating an occlusalgram that maps the location and magnitude of the occlusal contacts. They system may determine that the resin of the filling no longer extends beyond a desired height and that the occlusion between the upper and lower arch may be proper.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor. A computer-readable medium, such as a non-transitory computer readable medium, that may store instructions that, when executed by a computer perform any of the methods or processes described herein.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following numbered clauses.

Clause 1. A system for curing flowable dental material, the system comprising: an intraoral scanner configured to capture 3D data and having a probe at a distal end; a dental-material-curing light source configured to emit curing light from the probe; and a processor and memory comprising instructions that when executed by the processor cause the system to: receive a 3D model of the dentition of the patient from a dental treatment plan; receive scan data of the dentition of the patient; determine, while receiving the scan data, an area of illumination by the dental-material-curing light source with respect to a dentition of a patient, based on the received scan data.

Clause 2. The system of clause 1, wherein the intraoral scanner comprises a structured light scanner.

Clause 3. The system of clause 1, wherein the area of illumination of the dental-material-curing light source is in a known relationship with respect to a field of view of the intraoral scanner.

Clause 4. The system of clause 3, wherein the dental-material-curing light source is a plurality of light sources configured to selectively illuminate a plurality of areas of illumination.

Clause 5. The system of clause 1, wherein the instructions that when executed by the processor further cause the system to: determine that at least a first of the plurality of areas of illumination includes uncured dental material.

Clause 6. The system of clause 5, wherein instructions that when executed by the processor further cause the system to: selectively illuminate the first of the plurality of areas of illumination when the system determines that the at least the first of the plurality of areas of illumination includes uncured dental material.

Clause 7. The system of clause 6, wherein the instructions that when executed by the processor further cause the system to: determine that at least the first of the plurality of areas of illumination does not include uncured dental material.

Clause 8. The system of clause 1, wherein the dental-material-curing light source is used by the intraoral scanner to generate the scan data of the dentition of the patient.

Clause 9. The system of clause 3, wherein instructions that when executed by the processor further cause the system to: scan the detention of the patient with the intraoral scanner to generate the scan data.

Clause 10. The system of clause 9, wherein instructions that when executed by the processor further cause the system to: emit curing light from the dental-material-curing light source; while emitting curing light, receive the scan data; while emitting curing light, align the scan data with the 3D model of the dentition of the patient from the dental treatment plan, the dental treatment plan including information about curing locations and dosage.

Clause 11. The system of clause 10, wherein instructions that when executed by the processor cause the system to determine an area of illumination by the resin curing light source with respect to a dentition of a patient comprise instructions that further cause the system to determine the area of illumination based on the alignment of the 3D scan data with the 3D model of the dentition of the patient and the known relationship of the area of illumination of the resin curing light source with respect to the field of view of the 3D scanning system.

Clause 12. The system of clause 11, wherein instructions that when executed by the processor further cause the system to: determine that dental material that has not yet received a determined dose of curing energy is within the area of illumination of the dental-material-curing light source.

Clause 13. The system of clause 12, wherein instructions that when executed by the processor further cause the system to: activate the dental-material-curing light source when the dental material that has not yet received a determined dose of curing energy is within the area of illumination of the dental-material-curing light source.

Clause 14. The system of clause 13, wherein the dental-material-curing light source is configured to emit light within a range of 400 nm to 500 nm.

Clause 15. The system of clause 1, wherein instructions that when executed by the processor further cause the system to: provide feedback to the user.

Clause 16. The system of clause 15, wherein the feedback includes feedback regarding the curing status of the dental material.

Clause 17. The system of clause 16, wherein the feedback regarding the curing status of the dental material includes altering a displayed model of the dental material based on the curing status of the dental material.

Clause 18. The system of clause 16, wherein altering the displayed model of the dental material based on the curing status of the dental material includes modifying a color of the model at a location of the dental material based on the cured status of the dental material.

Clause 19. The system of clause 15, wherein the feedback includes feedback regarding visual, audio, or haptic feedback when the system estimates that the dental material is cured.

Clause 20. The system of clause 1, wherein instructions that when executed by the processor further cause the system to: determine that the area of illumination is unknown.

Clause 21. The system of clause 20, wherein instructions that when executed by the processor further cause the system to: while the dental-material-curing light source is activated, deactivate the dental-material-curing light source when the system determines that that the area of illumination is unknown.

Clause 22. The system of clause 20, wherein instructions that when executed by the processor further cause the system to: while the dental-material-curing light source is activated, deactivate the dental-material-curing light source to cease illuminating the first of the plurality of areas of illumination when the system determines that the at least the first of the plurality of areas of illumination does not include dental material that has not yet received a determined dose of curing energy.

Clause 23. The system of clause 1, wherein instructions that when executed by the processor further cause the system to: while the dental-material-curing light source is activated, deactivate the dental-material-curing light source to when the system determines that the at least the first area of illumination is outside an oral cavity of the patient.

Clause 24. The system of clause 16, wherein the curing status of the dental material is based on the dose of curing energy determined to have been received by the dental material and a predetermined dose to cure the dental material.

Clause 25. The system of clause 24, wherein the predetermined dose is predetermined based on a wavelength of light used to cure the dental material, the geometry of the dental material, and the light scattering and absorption properties of the dental material.

Clause 26. A method for curing flowable dental material, the method comprising: receiving a 3D model of the dentition of the patient from a dental treatment plan; illuminating the dentition of the patient with curing light emitted from a dental-material-curing light source of an intraoral scanner; receiving scan data of the dentition of the patient from the intraoral scanner; determining, while receiving the scan data, an area of illumination by the dental-material-curing light source with respect to a dentition of a patient, based on the received scan data.

Clause 27. The method of clause 26, wherein the intraoral scanner comprises a structured light scanner.

Clause 28. The method of clause 26, wherein the area of illumination of the dental-material-curing light source is in a known relationship with respect to a field of view of the intraoral scanner.

Clause 29. The method of clause 28, wherein the dental-material-curing light source is a plurality of light sources configured to selectively illuminate a plurality of areas of illumination.

Clause 30. The method of clause 26, further comprising: determining that at least a first of the plurality of areas of illumination includes uncured dental material.

Clause 31. The method of clause 30, further comprising: selectively illuminate the first of the plurality of areas of illumination when the system determines that the at least the first of the plurality of areas of illumination includes uncured dental material.

Clause 32. The method of clause 31, further comprising: determining that at least the first of the plurality of areas of illumination does not include uncured dental material.

Clause 33. The method of clause 26, wherein the dental-material-curing light source is used by the intraoral scanner to generate the scan data of the dentition of the patient.

Clause 34. The method of clause 28, further comprising: scanning the detention of the patient with the intraoral scanner to generate the scan data.

Clause 35. The method of clause 34, further comprising: emitting curing light from the dental-material-curing light source; while emitting curing light, receiving the scan data; while emitting curing light, aligning the scan data with the 3D model of the dentition of the patient from the dental treatment plan, the dental treatment plan including information about curing locations and dosage.

Clause 36. The method of clause 35, wherein determining an area of illumination by the resin curing light source with respect to a dentition of a patient includes determining the area of illumination based on the alignment of the 3D scan data with the 3D model of the dentition of the patient and the known relationship of the area of illumination of the resin curing light source with respect to the field of view of the 3D scanning system.

Clause 37. The method of clause 36, further comprising: determining that dental material that has not yet received a determined dose of curing energy is within the area of illumination of the dental-material-curing light source.

Clause 38. The method of clause 37, further comprising: activating the dental-material-curing light source when the dental material that has not yet received a determined dose of curing energy is within the area of illumination of the dental-material-curing light source.

Clause 39. The method of clause 38, wherein the dental-material-curing light source is configured to emit light within a range of 400 nm to 500 nm.

Clause 40. The method of clause 26, further comprising: providing feedback to the user.

Clause 41. The method of clause 40, wherein the feedback includes feedback regarding the curing status of the dental material.

Clause 42. The method of clause 41, wherein the feedback regarding the curing status of the dental material includes altering a displayed model of the dental material based on the curing status of the dental material.

Clause 43. The method of clause 41, wherein altering the displayed model of the dental material based on the curing status of the dental material includes modifying a color of the model at a location of the dental material based on the cured status of the dental material.

Clause 44. The method of clause 40, wherein the feedback includes feedback regarding visual, audio, or haptic feedback when the system estimates that the dental material is cured.

Clause 45. The method of clause 26, further comprising: determining that the area of illumination is unknown.

Clause 46. The method of clause 45, further comprising: while the dental-material-curing light source is activated, deactivating the dental-material-curing light source when the system determines that that the area of illumination is unknown.

Clause 47. The method of clause 45, further comprising: while the dental-material-curing light source is activated, deactivating the dental-material-curing light source to cease illuminating the first of the plurality of areas of illumination when the system determines that the at least the first of the plurality of areas of illumination does not include dental material that has not yet received a determined dose of curing energy.

Clause 48. The method of clause 26, further comprising: while the dental-material-curing light source is activated, deactivating the dental-material-curing light source to when the system determines that the at least the first area of illumination is outside an oral cavity of the patient.

Clause 49. The method of clause 41, wherein the curing status of the dental material is based on the dose of curing energy determined to have been received by the dental material and a predetermined dose to cure the dental material.

Clause 50. The method of clause 49, wherein the predetermined dose is predetermined based on a wavelength of light used to cure the dental material, the geometry of the dental material, and the light scattering and absorption properties of the dental material.

Clause 51. A non-transitory computer readable medium comprising instructions that when executed by a computing system, cause the system to: receive a 3D model of the dentition of the patient from a dental treatment plan; illuminate the dentition of the patient with curing light emitted from a dental-material-curing light source of an intraoral scanner; receive scan data of the dentition of the patient from the intraoral scanner; determine, while receiving the scan data, an area of illumination by the dental-material-curing light source with respect to a dentition of a patient, based on the received scan data.

Clause 52. The non-transitory computer readable medium of clause 51, wherein the instructions that when executed by the computing system, further cause the system to: determine that at least a first of the plurality of areas of illumination includes uncured dental material.

Clause 53. The non-transitory computer readable medium of clause 52, wherein instructions that when executed by the computing system, further cause the system to: selectively illuminate the first of the plurality of areas of illumination when the system determines that the at least the first of the plurality of areas of illumination includes uncured dental material.

Clause 54. The non-transitory computer readable medium of clause 53, wherein the instructions that when executed by the computing system, further cause the system to: determine that at least the first of the plurality of areas of illumination does not include uncured dental material.

Clause 55. The non-transitory computer readable medium of clause 53, wherein instructions that when executed by the computing system, further cause the system to: scan the detention of the patient with the intraoral scanner to generate the scan data.

Clause 56. The non-transitory computer readable medium of clause 55, wherein instructions that when executed by the computing system, further cause the system to: emit curing light from the dental-material-curing light source; while emitting curing light, receive the scan data; while emitting curing light, align the scan data with the 3D model of the dentition of the patient from the dental treatment plan, the dental treatment plan including information about curing locations and dosage.

Clause 57. The non-transitory computer readable medium of clause 56, wherein instructions that when executed by the computing system cause the system to determine an area of illumination by the resin curing light source with respect to a dentition of a patient comprise instructions that further cause the system to determine the area of illumination based on the alignment of the 3D scan data with the 3D model of the dentition of the patient and the known relationship of the area of illumination of the resin curing light source with respect to the field of view of the 3D scanning system.

Clause 58. The non-transitory computer readable medium of clause 57, wherein instructions that when executed by the computing system, further cause the system to: determine that dental material that has not yet received a determined dose of curing energy is within the area of illumination of the dental-material-curing light source.

Clause 59. The non-transitory computer readable medium of clause 58, wherein instructions that when executed by the computing system, further cause the system to: activate the dental-material-curing light source when the dental material that has not yet received a determined dose of curing energy is within the area of illumination of the dental-material-curing light source.

Clause 60. The non-transitory computer readable medium of clause 51, wherein instructions that when executed by the computing system, further cause the system to: provide feedback to the user.

Clause 61. The non-transitory computer readable medium of clause 60, wherein the feedback includes feedback regarding the curing status of the dental material.

Clause 62. The non-transitory computer readable medium of clause 61, wherein the feedback regarding the curing status of the dental material includes altering a displayed model of the dental material based on the curing status of the dental material.

Clause 63. The non-transitory computer readable medium of clause 61, wherein altering the displayed model of the dental material based on the curing status of the dental material includes modifying a color of the model at a location of the dental material based on the cured status of the dental material.

Clause 64. The non-transitory computer readable medium of clause 60, wherein the feedback includes feedback regarding visual, audio, or haptic feedback when the system estimates that the dental material is cured.

Clause 65. The non-transitory computer readable medium of clause 51, wherein instructions that when executed by the computing system, further cause the system to: determine that the area of illumination is unknown.

Clause 66. The non-transitory computer readable medium of clause 20, wherein instructions that when executed by the computing system, further cause the system to: while the dental-material-curing light source is activated, deactivate the dental-material-curing light source when the system determines that that the area of illumination is unknown.

Clause 67. The non-transitory computer readable medium of clause 65, wherein instructions that when executed by the computing system, further cause the system to: while the dental-material-curing light source is activated, deactivate the dental-material-curing light source to cease illuminating the first of the plurality of areas of illumination when the system determines that the at least the first of the plurality of areas of illumination does not include dental material that has not yet received a determined dose of curing energy.

Clause 68. The non-transitory computer readable medium of clause 51, wherein instructions that when executed by the computing system, further cause the system to: while the dental-material-curing light source is activated, deactivate the dental-material-curing light source to when the system determines that the at least the first area of illumination is outside an oral cavity of the patient.

Clause 69. The non-transitory computer readable medium of clause 61, wherein the curing status of the dental material is based on the dose of curing energy determined to have been received by the dental material and a predetermined dose to cure the dental material.

Clause 70. The non-transitory computer readable medium of clause 69, wherein the predetermined dose is predetermined based on a wavelength of light used to cure the dental material, the geometry of the dental material, and the light scattering and absorption properties of the dental material.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A system for curing flowable dental material, the system comprising:
an intraoral scanner configured to capture 3D data and having a probe at a distal end;
a dental-material-curing light source configured to emit curing light from the probe; and
a processor and memory comprising instructions that when executed by the processor cause the system to:
receive a 3D model of the dentition of the patient from a dental treatment plan;
receive scan data of the dentition of the patient;
determine, while receiving the scan data, an area of illumination by the dental-material-curing light source with respect to a dentition of a patient, based on the received scan data.

2. The system of claim 1, wherein the intraoral scanner comprises a structured light scanner.

3. The system of claim 1, wherein the area of illumination of the dental-material-curing light source is in a known relationship with respect to a field of view of the intraoral scanner.

4. The system of claim 3, wherein the dental-material-curing light source is a plurality of light sources configured to selectively illuminate a plurality of areas of illumination.

5. The system of claim 1, wherein the instructions that when executed by the processor further cause the system to:
determine that at least a first of the plurality of areas of illumination includes uncured dental material.

6. The system of claim 5, wherein instructions that when executed by the processor further cause the system to:
selectively illuminate the first of the plurality of areas of illumination when the system determines that the at least the first of the plurality of areas of illumination includes uncured dental material.

7. The system of claim 6, wherein the instructions that when executed by the processor further cause the system to:
determine that at least the first of the plurality of areas of illumination does not include uncured dental material.

8. The system of claim 1, wherein the dental-material-curing light source is used by the intraoral scanner to generate the scan data of the dentition of the patient.

9. The system of claim 3, wherein instructions that when executed by the processor further cause the system to:
scan the detention of the patient with the intraoral scanner to generate the scan data.

10. The system of claim 9, wherein instructions that when executed by the processor further cause the system to:
emit curing light from the dental-material-curing light source;
while emitting curing light, receive the scan data;
while emitting curing light, align the scan data with the 3D model of the dentition of the patient from the dental treatment plan, the dental treatment plan including information about curing locations and dosage.

11. The system of claim 10, wherein instructions that when executed by the processor cause the system to determine an area of illumination by the resin curing light source with respect to a dentition of a patient comprise instructions that further cause the system to determine the area of illumination based on the alignment of the 3D scan data with the 3D model of the dentition of the patient and the known relationship of the area of illumination of the resin curing light source with respect to the field of view of the 3D scanning system.

12. The system of claim 11, wherein instructions that when executed by the processor further cause the system to:
determine that dental material that has not yet received a determined dose of curing energy is within the area of illumination of the dental-material-curing light source.

13. The system of claim 12, wherein instructions that when executed by the processor further cause the system to:
activate the dental-material-curing light source when the dental material that has not yet received a determined dose of curing energy is within the area of illumination of the dental-material-curing light source.

14. The system of claim 13, wherein the dental-material-curing light source is configured to emit light within a range of 400 nm to 500 nm.

15. The system of claim 1, wherein instructions that when executed by the processor further cause the system to:
provide feedback to the user.

16. The system of claim 15, wherein the feedback includes feedback regarding the curing status of the dental material.

17. The system of claim 16, wherein the feedback regarding the curing status of the dental material includes altering a displayed model of the dental material based on the curing status of the dental material.

18. The system of claim 16, wherein altering the displayed model of the dental material based on the curing status of the dental material includes modifying a color of the model at a location of the dental material based on the cured status of the dental material.

19. The system of claim 15, wherein the feedback includes feedback regarding visual, audio, or haptic feedback when the system estimates that the dental material is cured.

20. The system of claim 1, wherein instructions that when executed by the processor further cause the system to:
determine that the area of illumination is unknown.
